(19) United States
(12) Patent Application Publication
(10) Pub. No.: US 2015/0273213 A1
(43) Pub. Date: Oct. 1, 2015

(54) Device and Multi-Function Apparatus for Applying Electrical Pulses to Bodily Tissue

(71) Applicant: L&S MEDTEC GBR, Erbach (DE)

(72) Inventors: Yuefei Liu, Neu-Ulm (DE); Hans-Jorg Simon, Erbach (DE)

(21) Appl. No.: 14/434,909

(22) PCT Filed: Oct. 24, 2013
(86) PCT No.: PCT/DE2013/000633

§ 371 Date: Apr. 10, 2015

(30) Foreign Application Priority Data
Oct. 27, 2012 (DE) 10 2012 021 136.8

(51) Int. Cl. A61N 1/05; A61B 17/34; A61N 1/08; A61N 1/39; A61B 5/06; A61M 11/00

(52) U.S. Cl. CPC A61N 1/0587; A61B 5/064; A61M 11/00; A61B 5/063; A61N 1/08; A61N 1/3968; A61B 17/3468

(57) ABSTRACT

Disclosed is a device (1, 30, 32, 38, 47, 55) for applying electrical pulses to pre-defined bodily tissue, which device can be placed on bodily surfaces of organisms. The device (1, 30, 32, 38, 47, 55) comprises at least one penetration device (9, 27, 33, 39, 48), which has at least one electrode (16, 31, 36, 42) and which is provided in order to penetrate into the organism after the device (1, 30, 32, 38, 47, 55) has been placed onto the bodily surface of an organism (22) and to assume an end position in which the electrode (16, 31, 36, 42) is in contact with the bodily tissue (25) of the organism (22), at least one position detection device (13), which is provided in order to detect a position of at least one part of the device (1, 30, 32, 38, 47, 55) lying against the bodily surface in relation to at least one part of the bodily tissue (25); and to subsequently check whether the detected position is suitable for transferring the penetration device (9, 27, 33, 39, 48) into the end position, and at least one signal output means (8) for emitting signals, the device (1, 30, 32, 38, 47, 55) being provided to emit at least one signal on the basis of a result of checking the detected position.

The present invention relates to a device that is abuttable against body surfaces of organisms for stimulating a given tissue with electrical impulses, and with a multifunctional apparatus having such device. The given tissue can be an internal muscle tissue or organ of a living organism that can refer to either human beings or animals or plants as well as to inner muscle tissues or inner organs of a dead organism like corpse or animal carcasses.

Usually, the electrical impulses are given to a dead organism for research purpose while these to a living organism mainly for medical treatments. As a known example of a device used due to medical reasons is a pacemaker that stimulates the heart muscle with electrical impulses and thus initiates the cardiac contraction. Furthermore, it is known that to treat heart arrhythms a defibrillator with large surface area electrodes placed on the surface of the chest wall of a patient can be used by sending electrical impulses through the chest wall to the heart muscle. Defibrillators play an important role in the emergency medicine. Nowadays, a number of defibrillators are available not only for the hospitals and ambulance but also for diverse public places where in an emergency case the defibrillators are operated by a lay person or layman.

Especially in the case of asytole or aborted systole, i.e. standstill of the heart without electrical and mechanical action, that causes death without immediate treatment, the defibrillator is effectiveless. In contrast to the ventricular fibrillation in which the defibrillation through a defibrillator serves as a specific and effective measure, the asystole is responsible for a sudden cardiac death to a greater degree. In addition, there is a lack of a specifical treatment for asystole. At present, an extrakorporeal cardiac massage is a unique possibility to handle the asystole out of hospital. The extrakorporeal cardiac massage requires to a certain extent skill and can be only done by a lay person with difficulty. Even for an experienced expert, the extrakorporeal cardiac massage has a very low successful rate below 5% and in general, frequent critical complications including injury of inner organs or rib fractures have been observed, which lead to significant impairment of the health of the patients. For these reasons, an intracardiac pacemaking is in the emergency treatment of asystole particularly desirable, which is unfortunately impossible without a previously placed electrode in the heart.

Nowadays, there are different procedures known, through which the diverse medical instruments can be introduced into and positioned at the inner tissues or organs or in the near of an inner organ of a living body or a died organism. An example is a catheter made of flexible material (from U.S. Pat. No. 6,309,370 B1), which is designed for automated or manual placement through an artery into the heart chamber. For this purpose, the catheter is mounted with a position sensor for determination of the position of a distal end of the catheter in relation to the heart and to the heart chamber. The suitable position sensor for this use is published in WO 96/05768 A1.

All the known procedures for introduction of the medical devices into the organisms are time-demanding and can be performed almost only by the medical professionals with the corresponding equipments. To get an electrode into the heart so that an intracardiac stimulation by the pacemaker is only possible through a cumbersome and time-demanding invasive procedure. For this reason, at present there is no possibility in the emergency care of asystole when the time is a critical factor, to get an intracardiac pacing, neither by the experienced physician nor by the lay persons.

Based on the situation mentioned above, it is a problem to provide a device and a multifunctional apparatus having such device that can be operated by even a medically unexperienced lay person in order to quickly supply electrical impulses to the organisms.

The problem is solved by the device having the features of claim 1, and by the multifunctional apparatus having the features of claim 13. Preferred embodiments are the objects of the dependent claims.

In order to electrically stimulate a given tissue of an organ, for instance an inner organ like the heart, the device according to the present invention is to be applied by a user to the surface of the body, possibly near the final destinational position of this device at first. Then, through the position determination apparatus the position of at least one part of the device abutting against the body surface is determined in relation to the organ or to a part of the organ that is to be electrically stimulated. This part of the device is preferably the puncturing apparatus or a part of it, e.g. a front-end portion of the puncturing apparatus or an electrode. Once the organ that is to be electrically stimulated is the heart, the position determination apparatus can determine the position of the puncturing apparatus or a part of it in relation to a heart chamber as an example. The determined position will be checked, and depending on the result of this check the signal output means will give signal for this. If the determined position through the position determination apparatus is suitable for the puncturing apparatus getting to the final position, the signal output means gives for instance at least the first signal. Furthermore, the signal output means is designed besides the first signal that indicates the suitable position for the puncturing apparatus, to give at least the second signal when the acquired position is recognized as unsuitable, whereby the two signals are different from each other. Is the determined position recognized as unsuitable, there will be, dependent upon the embodiment of the device of the present invention, no signal or at least the second signal will be given, which indicates to the user that it is impossible for the puncturing apparatus at the current position to get into the organism and to contact the tissue, and a displacement of the device to the other site of the body surface is necessary. Alternatively, at least a corresponding signal can be given in the case that the determined position is recognized as unsuitable, while no signal will be given as long as the determined position is regarded as suitable. During the displacement of the device guided by the position determination apparatus each new position of the device or a part of this is assessed, dependent upon whether the new position is suitable or not, a default signal from the signal output means will be or will be not given. When the signal output means gives once again a signal as unsuitable position, the user has to search for another position and to change the position of the device where it is possible for the puncturing apparatus to get to the final destinational position. This procedure will be repeated so long till a suitable position is found and the user is given the signal by the signal output means that at the current position the apparatus can be principally introduced into the organism such that the electrode can contact the given tissue. After setting the device at the final position and establishment of the contact of the electrode to the given tissue, this tissue can be stimulated with electrical impulses, whereby the electrode delivers the electrical impulses to the tissue. In this way, the device of this invention can be particularly simply operated, by even a non-medical lay person. The present invention allows a medical lay person to correctly place the device on the body of a patient without difficulty and to inject the intracardiac apparatus into the body and to establish the situation that the electrode contacts the desired tissue so that the electrical impulses can be provided to the tissue.

Principally, the kind of the signals sent by the signal output means is not essential. In this regard, the signals can be the simple light-emitting diodes, a screen or a display or a touch sensitive screen or a touch screen or a speaker. Accordingly, the signals can be optical or lighting or blinking or display of text or an audible signal or a linguistical or a vibration or a combination of two or more of these or others.

Furthermore, an audible and/or a visual guidance may be provided for the device of the present invention. Furthermore, the linguistical instruction or the user manual for operating the device is possible.

The device of the present invention has advantageously a flat cover plate at an end which during operation of the device is opposite to the body surface. A flat cover plate allows the device to be used for the cardiac massage provided that the treatment of asystole by the device remains unsuccessful. For this the puncturing apparatus can be taken out of the heart or the body before the cardiac massage is performed; certainly, this should be done by a physician. It is also possible that in order to improve the efficacy of the cardiac massage the heart can be stimulated through the electrical impulses during the cardiac massage.

Preferably, the determined position is recognized as suitable by the position determination apparatus if the determined position is within at least one of preset position range and/or if a route from an intended injection site for the puncturing apparatus to the tissue is free of the obstacles. The inclusion of the determined position in the given positioning range is considered as a criterium whether the determined position is principally suitable or not for the puncturing apparatus getting into the organism for the device of the present invention. Hereby the single positions in the positioning area are shown that when the device or a part of the device takes these positions, the puncturing apparatus is in the situation to perform its defined function and to get into the organism, so that the final destinational position can be attained where the electrode gets contact to the intended tissue. It is easy to compare a position determined by the position determination apparatus with the intended position area in order to ascertain whether this position is or is not located in the intended position area. In the case that this position is fallen in the intended position area, this position is regarded as suitable and the signal output means reacts correspondingly. Is this position not fallen in the intended position area, it is regarded as unsuitable, which is shown by the signal output means.

As a general rule, dependent on the kinds of the intended tissues or inner organs of an organism that is to be stimulated by electrical impulses, the number of the suitable positions on the body surface for the intracardiac device to get to the final position is different. Thus, there are different position areas for different inner organs of an organism. Therefore, a device has advantages by being given the default positioning areas when the single organs of the certain organisms are defined, for example, stored in the data form on a save medium. These given positioning areas can be obtained beforehand based on the typical or the average measurements of the involved organism, which are derived from the determination of many individual organisms. Furthermore, the device has advantageously a select option with which the user selects a given positioning area, depending on the organ to be stimulated with electrical impulses, which is taken for comparison with that determined by the position determination apparatus.

Although it is not essential for the primary position of the device on the surface of the body in relation to the given tissue or organ, it is preferably to place the device relatively nearby the desired organ of an organism in order to accelerate the positioning procedure. To indicate to the user the site of the surface of the body that is possibly suitable for placement of the device where the determined position within the positioning area so that the user probably locates the device on the site even at the first try, the device displays a graphic picture, through which the area suitable for the placement of the device on the body of the organism is indicated.

Alternative or additional to the default positioning area there are also other criteria included for the decision whether the determined position is suitable for the puncturing apparatus getting to the final position, e.g. existence of a bone tissue between the site for injection of the puncturing apparatus and the given tissue of the body. Because the existence of a bone tissue between the given injection site and the intended tissue of the body prevents the penetration of the puncturing apparatus, the puncturing apparatus is preferably placed at such a site of the organism where the gateway to the default tissue of the body possibly is not hindered by the bone structures and other obstacles like tumors. Only the determined position without such obstacles is considered as suitable for the puncturing apparatus getting into the organism, otherwise they are recognized as unsuitable.

The puncturing apparatus can be in the simplest case a canula made of metal or plastics. In this invention, it is possible that the apparatus gets the contact to the tissue either on its way to the final position or after its placement at the final position. Hereby this apparatus can be punctured at least partly into the tissue or organ or penetrate an outer wall of an organ in order to get into the chamber of the organ at least partly or completely. For the contact between the electrode and the organ, the simplest way can be a pointwise touch to the organ. When the electrode builds a contact area, it can also be placed on the outer side of the organ. Once the electrode is designed only for pointwise touch or flat contact of the outer side of the organ, it has the advantage when the apparatus of the present invention is equipped with a mechatronics that provides a dynamic adaptation or tracking the electrode to contact the organ when the organ contracts or moves. In the other cases, the electrode of the present invention penetrates the outer area of the organ and gets partly or completely into the organ. When the organ has at least one inner chamber, the puncturing apparatus can for instance penetrate the outer wall of the organ and gets into the chamber where the electrode is placed on the inner wall side of the chamber to establish the contact between the electrode and the organ. Hereby a part of the electrode straddles into the outer wall of the organ to get an additional contact within the outer wall besides the contact to the inner wall at the puncture site so that the delivery of the electric power to the organ can be improved due to the bigger contact area. Any combinations of this functional design are possible. For example, for stimulating the human heart with electrical impulses the construction of the apparatus of the present invention is especially preferred to allow the puncturing apparatus to penetrate the heart wall and to get into the heart chamber, then the electrode primarlly folded together extends out of the puncturing apparatus and evolves in the cavity, thereafter the puncturing apparatus is drawn back till that the unfolded electrode contacts to the inner side of the heart chamber. The position of the puncturing apparatus is now the final position when the unfolded electrode is within the heart chamber and contacts the heart.

The apparatus of the present invention, particularly the electrode can be supplied with power, through which the electrical impulses can be applied to the tissue, there are several power sources available or at least one connection for the electrode. Through this connection the apparatus can be connected to an external energy or power source. For instance, the apparatus can be designed for operation mode of electricity network and connected to an electricity power supply. Furthermore, the apparatus can have recess where the power connection is arranged and for which one or more batteries or accumulators or other power sources are available.

The device of the present invention contains preferentially at least one sensor and/or at least one sensor array and/or at least one ultrasonic sensor and/or at least one infrared sensor and/or at least one force sensor and/or at least one depth sensor and/or at least one magnetic position sensor and/or at least one laser sensor and/or at least one laser-emitting diode and/or at least one gas sensor and/or at least one skin resistance sensor and/or at least one sensor for impedance measurement. Principally, not only the position determination apparatus but also the puncturing apparatus can be equipped with a sensor. It is also possible that the sensors being implemented can be used by both the apparatuses. Thus, in order to determine the position of at least one part of the apparatus in relation to at least one inner organ of the organism, the position determination apparatus can be equipped with one or more ultrasonic sender and receiver as ultrasonic sensors or with infrared. Similarly, the puncturing apparatus can be equipped with one or more ultrasonic sender and receiver or ultrasonic sensor or infrared sensor that help monitor way of the puncturing apparatus to the final position. Alternatively, the ultrasonic sensors or infrared sensors can be taken so that they can not only determine the position of at least one part of the apparatus in relation to at least one part of the inner organs of the organism through the position determination apparatus but monitor the penetration of the puncturing apparatus into the organism as well. Through the ultrasonic senders and receivers or ultrasonic sensors and infrared sensors, the inner organs and respectively the parts of the inner organs, e.g. hollows as well as the process or the progress of the penetration of the puncturing apparatus into the organism can be displayed in pictures. When the device of the present invention has a display or is connected to a display, the pictures derived from such sensors can be shown on the screen and make it easier for the user to operate and control the puncturing apparatus during the penetration into the organism as well as to control the contact of the electrode to the tissues or organs of the body. Hereby, particularly an infrared sensor or an ultrasonic sensor is intended for determining whether the position of at least one part of the apparatus that placed on the body surface in relation to at least one part of the tissue, is suitable for the puncturing apparatus to get to the final position, that means, an ultrasonic sensor is intended for determining the depth till the puncturing apparatus gets into the organism. In order to control the process of the penetration of the puncturing apparatus or the electrode into the organism or the tissues or organs, the force sensors are preferably to be constructed as a part of the puncturing apparatus. For example, penetrating the skin and the outer muscular fascia of an organism generally needs greater force than that for an organ. Based on the force respectively needed during the penetrating procedure of the puncturing apparatus getting into the organism, it can be recognized that the puncturing apparatus has penetrated the outer muscle fascia and got into the organ. Using the depth sensors, the position of the puncturing apparatus is detected by the depth where the puncturing tool has got into the organism. Contrastly, by using a magnetic position sensor, the actual position of the puncturing apparatus within an organism can be directly detected. The suitable sensors as force sensors, ultrasonic sensors and infrared sensors advantageously provide additional measurements that determine whether there are the bone structures between the intended injection site for the puncturing apparatus and the tissue or organ, these structures would prevent the penetration of the puncturing apparatus. The gas sensors, particularly the air sensors, make the detection of gases and air in the puncturing apparatus possible, and they monitor the air that gets in the puncturing apparatus. If such sensors detect the existence of the air in the puncturing apparatus, the device of the present invention gives a corresponding signal to the signal output means and to block the penetration of the puncturing apparatus even if the position is previously considered as suitable by the position determination apparatus, so that it can be avoided that the gases or air get into the organism and cause the health problems there.

Preferably, the puncturing apparatus comprises at least one cutting means for cutting the organism in order to make it easier for the puncturing apparatus to get into the organism. Principally, all the means are suitable when they can locally slit or separate the organism and allow the puncturing apparatus to get into the organism. In this respect the organism can be slit prior to or during the penetration of the puncturing apparatus. The cutting means can be especially a cutting edge or a blade that is mounted at the front end of the puncturing apparatus and penetrates at first the organism, so that during the penetration of the puncturing apparatus into the final position the organism can be slit directly before the puncturing apparatus, which makes the penetration of the puncturing apparatus possible. The cutting means can be also a pointy or wedge-formed front end of the puncturing apparatus, with which the puncturing apparatus at first penetrates into the organism.

In an advantageous embodiment of the device of the present invention, the puncturing apparatus has at least two elements that are slidable in a telescope-like way into one another and apart. Principally the puncturing apparatus can also have three or more such telescope-like elements that slide into one another and apart. Such telescope-like elements allow the particularly simple injection of the puncturing apparatus into the organism. Preferred is that the electrode is mounted at or in the front end of the elements, especially the one of these elements that penetrates farthermost into the organism, which is usually the innermost of the sliding elements. Particularly preferential is the outermost one of these elements with a stable husk that is used to penetrate the outer layer of the organism like the skin, the muscular fascia and gets into the organism until it with its front-end just touches or is near the outer side of an organ. In this position of the husk, one or more elements contained in the telescope-like husk can get out of the husk and contact directly the organ or get into the organ or penetrate the outer wall of the organ. Because the resistance of the organ wall is usually higher than that of the muscle tissue, these telescopic-like elements in the husk or pushed in outermost elements can be less stable or robust than that of the outermost element.

There are possible embodiments of the present invention, in which the electrode covers or builds at least a part of an outer surface of the puncturing apparatus; and/or the puncturing apparatus comprises at least one foldable means whereby the electrode covers or forms at least a part of the outer surface of the foldable means; and/or the puncturing apparatus comprises at least one expandable means whereby the electrode covers or forms at least a part of the outer surface of the expandable means; and/or the apparatus comprises at least an extendable element in which the electrode covers or forms at least a part of the outer surface of this extendable element. The electrode is preferably constructed by a metal mesh that builds or covers the corresponding part of the outer surface. The foldable means is preferably constructed in a folded status hosted in a cavity of the puncturing apparatus. For example, the foldable means is made as a foldable umbrella-like construction likewise a mesh that can be closed and opened via sprint joints as umbrella. In one embodiment of the present invention, the foldable means gets out of the cavity of the puncturing apparatus, folds out, and with its open status takes a relatively great area and docked at the outer side of an organ so that the electrode covering a part of the outer surface of the foldable means contacts the organ, and the puncturing apparatus takes the final position. For another embodiment of the present invention, the puncturing apparatus penetrates an outer wall of the organ and gets into the chamber of the organ; afterwards the foldable means gets out of the cavity of the puncturing apparatus and folds out. With its open status, the puncturing apparatus withdraws a bit back again until the opened foldable means is attached to the inner wall of the chamber and the electrode covering a part of the outer surface of the foldable means now contacts the inner wall of the organ. Upon this, the puncturing apparatus has reached its final position. Both these embodiments of the foldable means described here are also possible for the expandable means instead of the foldable means, where the expandable means correspondingly at first in a closed status arranged in the cavity of the puncturing apparatus gets out of the cavity and expands in a relative nearness of an organ or within a chamber of the organ in order to dock then either at the outer side of the organ or at an inner surface of the chamber of the organ, the electrode covering a part of the outer surface of the expandable means contacts the organ. Hereby the expandable means can be constructed as the known cardiac catheters. For expansion of the expandable means, it is advantageous for the apparatus to have among others an extending fluid pipe through the puncturing apparatus, which supplies necessary fluid to the inner chamber of the expandable means for expanding. This fluid can be respectively water or air. Furthermore, the apparatus advantageously comprises a fluid reservoir for storage of the fluid or comprises a connector for connection to a fluid reservoir, through which the fluid can be supplied to the apparatus and finally the expandable means. The described embodiments of the present invention in which the foldable means or the expandable means within the chamber of the organ folds out or expands and then attach to the inner wall of the chamber, are particularly preferred because the foldable and expandable means function as barbs and the slipping of the electrode as well as the puncturing apparatus out of the chamber is prevented; therefore a good contact between the electrode and the organ is ensured. To remove the electrode or the puncturing apparatus from the organism or the organ, the foldable means will be folded in a closed status and the expandable means is drained from the fluid, respectively. This leads to a closed status of the means and the respective means can be fed into the cavity of the puncturing apparatus, and now the puncturing apparatus can be comfortably removed from the organ and the organism.

A further embodiment of the present invention has advantageously at least one drug feed for supplying the tissue with at least one drug, and at least one drug reservoir coupled to the drug feed for receiving at least one drug, and/or at least one drug inlet coupled with the drug feed for getting at least one drug from outside the device. For example, there can be one, two, three or more drug reservoirs with one drug each; or one, two, three or more drug inlets, each for a drug, into the apparatus. The drug in the drug reservoir can be pneumatically pressed, e.g., through a pneumatic-driven piston, from the drug reservoir into the drug feed; or alternatively, there is at least one pump, preferably controllable, provided to get the drug from the drug reservoir and to bring it to the drug feed, so that the tissue can be supplied with medicine. Correspondingly, a drug inlet can be preferably a steerable pump to take drugs from outside of the apparatus into the drug feed. Furthermore, a drug inlet can be a connector for connection to an external drug feed for the drug, e.g. a tube or an external drug reservoir. With this embodiment of the present invention, the drugs like adrenaline, atropine or bicarbonates can be supplied to the tissue or an organ. These drugs will be chosen particularly when the organ is the human heart. The drug feed can have one extendable feeding tube that is the inner of the puncturing apparatus. Furthermore, the drug feed can have one or more nozzle-like diffusers that spray the medicine upon the organ, particularly the inner of a chamber of the organ. Provided that the puncturing apparatus possesses, for instance, an expandable part like a balloon, this can be such as a membrane or shell made of silicon or polyethylene; and an expansion element with the expanded status at least at the side turning away from the inner wall of the chamber of the organ, which is at least partly concealed by the membrane or the shell. This membrane can be constructed with a number of diffusers or perforations, through which the drug in the gap between the membrane and the expansion element can flow from the gap to the chamber of the organ; hereby the drug is sprayed out via a number of nozzles.

Additionally, the device of the present invention is preferred to have at least one movable abutting element for abutting the device against body surfaces of organisms. Thus, the device can have one or more movable abutting elements with respective levels or contact surfaces that are tied to the housing of the device in more or less scattered positions or neighbouring from each other. Furthermore, there can be one or more supporting elements, for example, pin-like supporting elements, as installation elements that can be arbitrarily formed, and particularly can be constructed more or less as spiky or also blunt with flattered or rounded cross sectional face. Especially, the installation elements can be formed as movable and/or tilting against each other in order to adapt to the body surfaces with different forms and maintain reasonable placement of the device on the organism. For this purpose, an adhesive fixation to the body surface can be constructed for the device in order to prevent the apparatus shifting.

For one embodiment of the device of the present invention, the depth to which the puncturing apparatus gets into the organism is adjustable. For example, this depth can be adjusted or given manually by a user, which can be subsequently corrected, e.g. by a physician. Advantageously, the device has a display such as a monitor for depth, which can show the individual given depth and/or the depth that the apparatus has reached during the penetration procedure. For the other embodiments of the device of the present invention, there is at least one given depth value up to which the puncturing apparatus and/or the electrode gets into the organism. This kind of the embodiment form has the advantage that during the procedure of the penetration of the puncturing apparatus, no measurements are needed and the penetration itself must not be steered because it is determined from the beginning how deep the puncturing apparatus respectively the electrode gets into the organism. A depth value can be, for instance, simply be given by the distance to which the puncturing apparatus can slide out of the device housing at maximum. For such simple embodiments of the device, the puncturing apparatus will be put into the final position once being triggered based on the preset. There may also be more different depth values given, depending not only on the kind of the tissues to be stimulated with electrical impulses but also on the size of the organism. Depending on the tissues that are electrically stimulated and the size of the organism, an individual depth value, either manually or automatically via the control unit will be chosen, and the puncturing apparatus gets so far at this depth into the organism without that this procedure must be controlled.

Because in such devices the penetration depth to which the puncturing apparatus gets into the organism is preset, the puncturing apparatus penetrates into the same depth for all the organisms. This is especially critical for such preset penetration of the puncturing apparatus to get the final position in the living organisms because the individual difference with regard to the anatomy of the organisms are not considered, and the organisms herewith can be injured. In addition, a functional failure of the puncturing apparatus can occur. Therefore, the device of the present invention preferably comprises a control unit, which is configured for controlling the delivery of the electrical impulses to the tissue and/or the checking the determined position and/or the output of signals and/or the penetration of the puncturing apparatus into the organism and/or the transfer of the puncturing apparatus to the final position and/or the contact of the electrode to the tissue and/or the folding-out of the foldable means and/or the expansion of the expandable means and/or the supply of drugs to the tissue and/or the positioning adjustment of the abutting elements against an outer form of the body surface. Such a control unit allows a largely automated process of the apparatus. The control unit can advantageously be a microprocessor. The control unit can particularly be a part of the position determination apparatus and responsible for checking the determined position. In particular for contacting the heart of living patients with electrode, the penetration of the puncturing apparatus into the organism and the transferring of the puncturing apparatus to the final position, this kind of the control unit can control reliably and automatically without dependence on the user. Another embodiment of the present invention comprises an additional wireless communication connection between the apparatus and its distantly arranged control unit or a control device, so that the transferring of the puncturing apparatus to the final position as well as any further and even other functions of the device can be controlled at a distance through the control unit or the control device as well as a computer. In particular, the wireless communication connection can be a WLAN connection or a Bluetooth connection.

Advantageously, the device of the present invention comprises as housing, wherein the puncturing apparatus can be decoupled from the housing. When the device is applied for the primary treatment of asystole in an emergency, this device should usually remain at the patient, or the puncturing apparatus should not be separated from the housing of the device until the medical care is warranted. In exceptional cases, a separable or decouplable puncturing apparatus may have the advantage because to fix the relatively smaller puncturing apparatus is easier than to fix the relatively big device against slipping. After the successful first aid for the patient on site, a separation of the puncturing apparatus from the device housing can make the transport of the patient to a hospital via a rescue vehicle. The decoupled puncturing apparatus can for instance be connected to a device within the rescue vehicle, which can also stimulate the heart of the patient with electrical impulses during the transport if necessary. By arriving in the hospital, the puncturing apparatus can be then carefully removed from the heart and further from the body of the patient by a physician.

In the case of successful treatment of the asystole by the device of the present invention, there are contractions of the heart muscles, which bears the risk of slipping of the puncturing apparatus or of detaching of the puncturing apparatus from the heart. In this situation, the contact of the electrode to the heart and then the supply of electrical impulses to the heart can be broken. Therefore, the device of the present invention is preferably constructed suitable for tracking the final position of the puncturing apparatus when the tissue performs movements. For tracking the final position of the puncturing apparatus, the device has for example a fixing and pulling-back mechanism or one or more mechanic, mechatronic, pneumatic or hydraulic elements or components or a fixing or pulling-back mechanism with at least such a kind element or component. In a device tracking the final position of the puncturing apparatus, an optimal contact of the electrode to the tissue is assured, even when the tissue brings about movement, e.g. contractions or expansion movements.

Principally, the device of the present invention can be constructed autonomously and compactly, so that it is portable and easy to operate. On the other hand, the device of the present invention can be a part of a system or a multifunctional apparatus. In particular, a multifunctional apparatus with the device of the present invention is preferred to include at least one defibrillator and/or at least an electrode of a defibrillator and/or at least a cardiac pacemaker. With the functions of the device of the present invention, such kind of the multifunctional apparatus advantageously unifies the functions of defibrillators and pacemakers. When this kind of multifunctional apparatus instead of the nowadays generally used defibrillators is provided, it is possible in an emergency case for everyone and even a medical lay person or layman to directly and immediately handle the asystole after unsuccessful applied defibrillation, this is done by the one and the same device. Herewith, not only the death rates due to asystole in emergency enormously decrease, but the frequently happened injuries resulted from chest compression can be completely avoided as well.

The present invention is described in more details as follows based on the preferential examples of the embodiments with the help of the figures. It is shown:

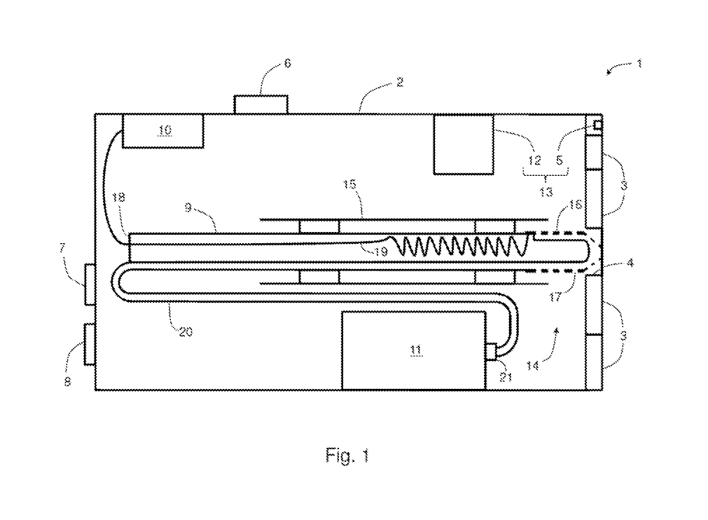

FIG. 1 a simplified cross section through an emergency aid model;

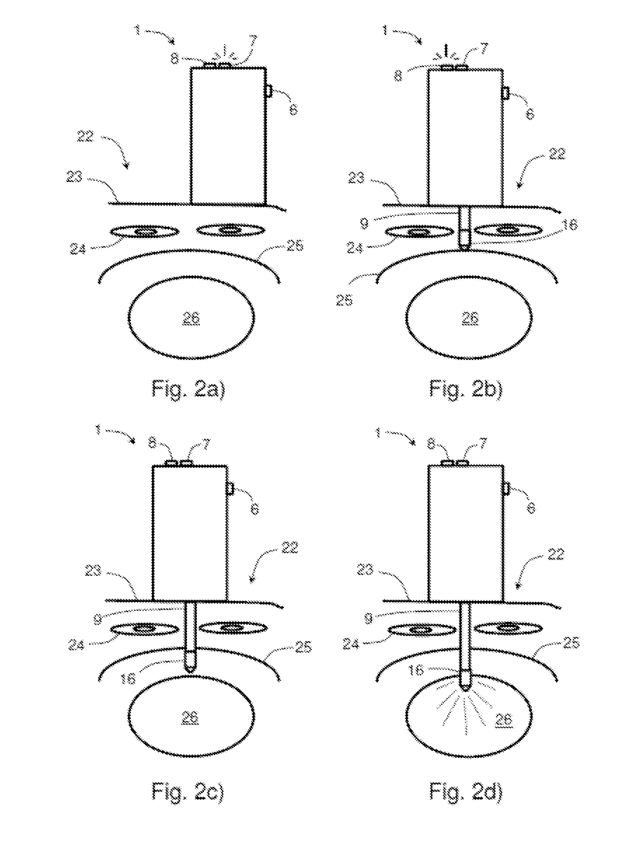

FIG. 2a)-d) the functioning of the emergency aid model in FIG. 1;

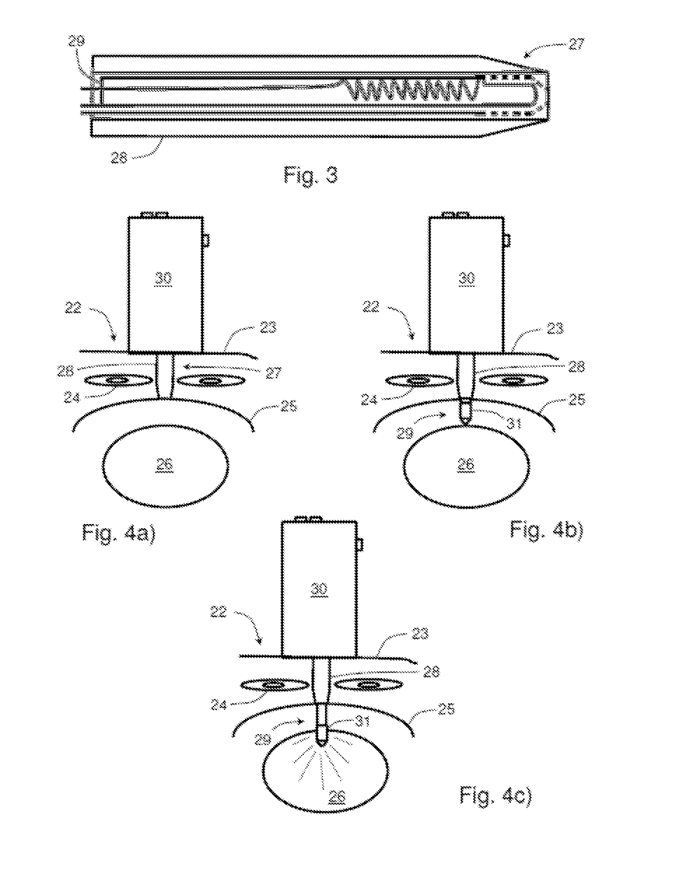

FIG. 3 a simplified cross section through a puncturing apparatus;

FIG. 4a)-c) the functioning of an emergency aid model with the puncturing apparatus in FIG. 3;

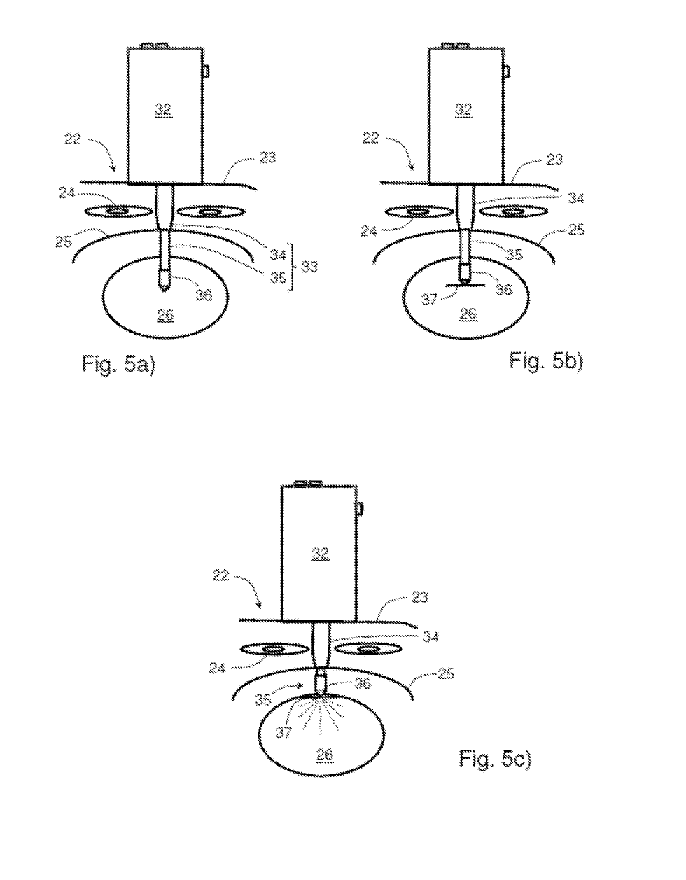

FIG. 5a)-c) the functioning of an emergency aid model with an alternative puncturing apparatus;

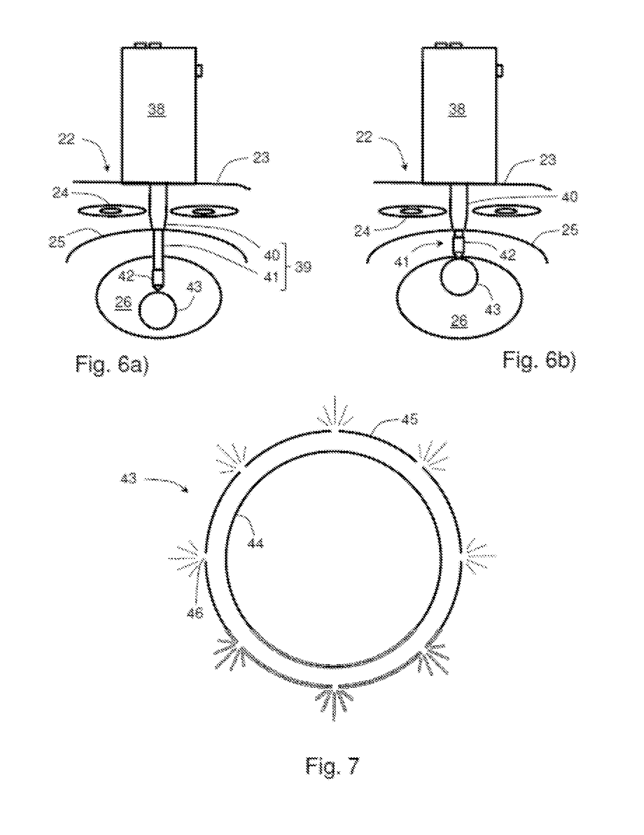

FIG. 6a)-b) the functioning of an emergency aid model with a further alternative puncturing apparatus;

FIG. 7 a cross section through the balloon of the emergency aid model in FIG. 6a)-b);

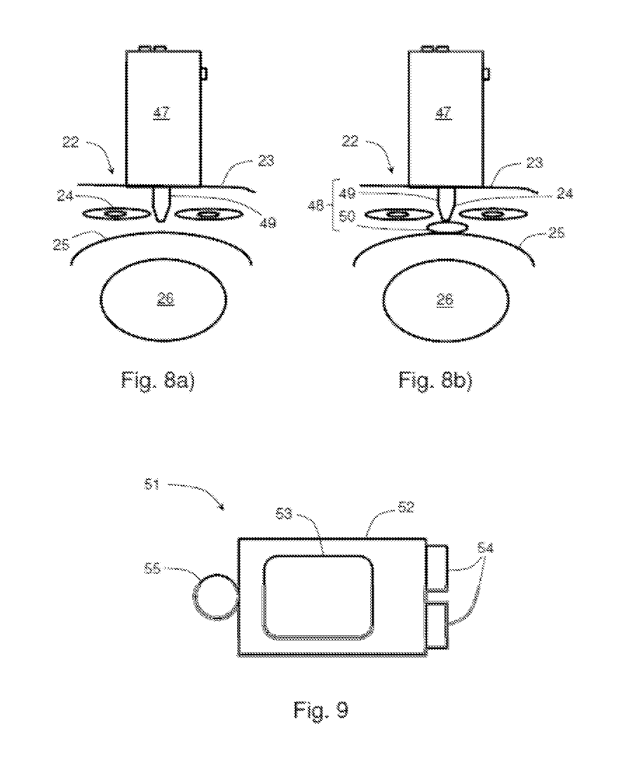

FIG. 8a)-b) the functioning of a further emergency aid model;

FIG. 9 a multifunctional emergency equipment with an emergency aid model;

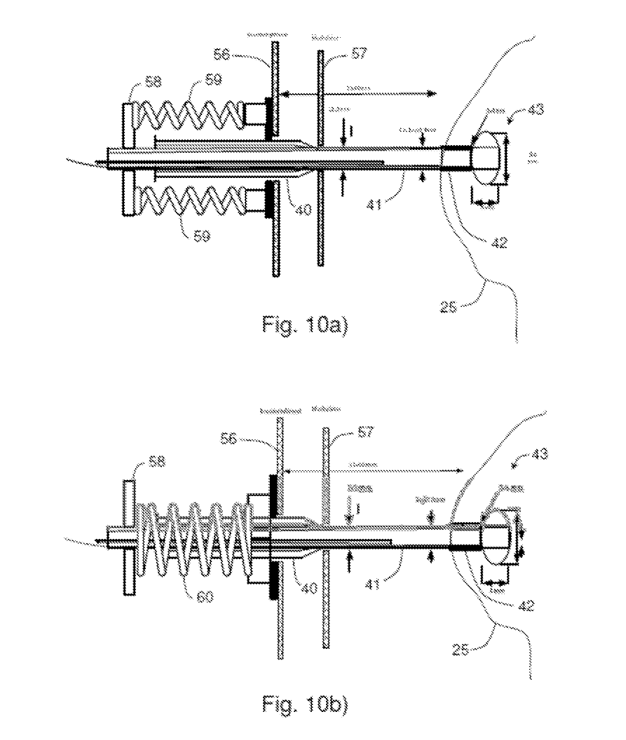

FIG. 10a)-b) tracking apparatuses for puncturing apparatus;

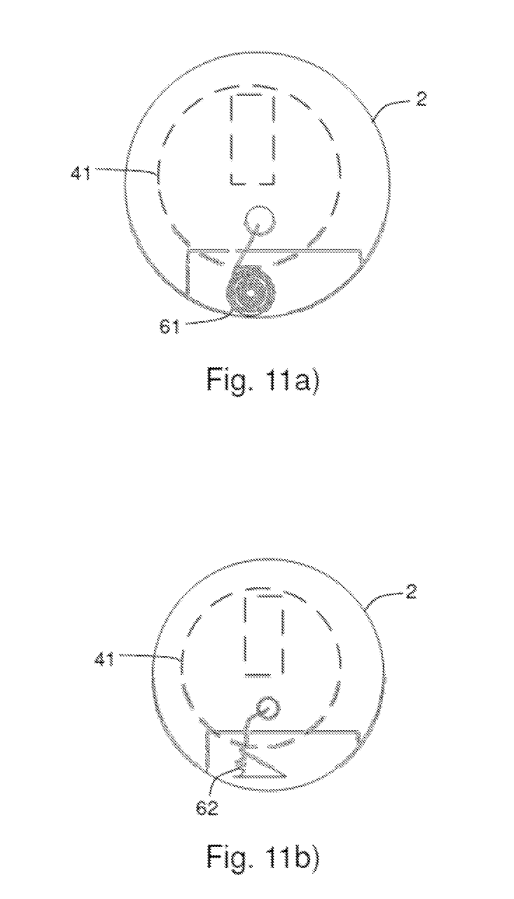

FIG. 11a-b) tracking apparatuses with different springs.

In the FIG. 1 it is shown a strongly simplified, not true to scale, and due to the better description a strongly schematic illustration of an emergency aid model 1 for stimulation of the human heart with electrical impulses, which essentially corresponds the device of the present invention.

The emergency aid model 1 comprises a cylindrical housing 2, the base area with a circular form of which is built with a number of movable installation elements 3, relatively to each other as well as related to the housing 2, where the base area contains a central-omitting opening 4, and a sensor array 5 that can refer to a combination of an infrared sensor and an ultrasonic sensor and be implemented in the base area. On the lateral surface of the housing 2 there is a manually-operating activation button 6 installed, whereas on the mainly flat cover area of the housing 2 there are a red light-emitting diode 7 and green light-emitting diode 8 installed. Additionally, the emergency aid model 1 has an oblong, throughout, hollow puncturing apparatus 9, a battery 10, a drug reservoir 11 and a control unit 12 in form of a microprocessor, all of these components are implemented in the housing 2. Hereby, the control unit 12 is connected with the sensor array 5 for data communication and builds together with the sensor array 5 a position determination apparatus 13. For the reason of the clarity, neither is the connection between the control unit 12 and the sensor array 5 is shown in the FIG. 1, nor the connection between the control unit 12 and the other components of the emergency aid model 1 for the same reason. As described as follows, these components are controlled by the control unit 12.

The principally hollow puncturing apparatus 9 that can be a metal cannula, extends centrally along the long axis of the housing 2, whereby a front end part 14 of the puncturing apparatus 9 facing to the mainly tapering end of the openings 4 is aligned with or protruded in the openings 4, without the penetration of the openings. A mechanics 15 is included for shifting of the puncturing apparatus 9 along the long axis of the housing 2. Up to certain degree, the mechanics 15 can also tilt the puncturing apparatus 9, so that the long axes of the housing 2 and the tilted puncturing apparatus 9 can respectively build an angle. Through the mechanics 15, the puncturing apparatus 9 with its front-end part 14 can forwards shift throughout the housing 2. The front-end part 14 of the puncturing apparatus 9 is covered with a metal mesh that serves as the electrode 16. Furthermore, the front-end part 14 is perforated and has a number of diffusers 17 that extend through not only the wall of the puncturing apparatus 9 but the electrode as well. An electrical cable 19 that extends itself within the puncturing apparatus 9 and is sectionwise wound as well as extends out of a rear end 18 of the puncturing apparatus 9, connects the electrode 16 and the battery 10, so that the electrode can be supplied with electrical impulses by the battery 10. Additionally, in the inside of the puncturing apparatus 9 there is a tubular fluid pipe 20 that protrudes out of the end 18 of the puncturing apparatus 9 as the electrical cable 19 and is connected via a pump 21 to the drug reservoir 11 filled with liquid drug. Here the fluid pipe 20 has besides the puncturing apparatus enough length and flexibility in order to allow the puncturing apparatus 9 getting out of the openings 4, so that the connection of the fluid pine 20 to the pump 21 is not prevented. In the front-end part of the puncturing apparatus 9, the fluid pipe 20 in the inside of the puncturing apparatus 9 merges into the gap between the throughout diffusers 17 and the outside of the puncturing apparatus 9, and this gap is built in the puncturing apparatus by the diffusers 17 covered with perforations and connected to these diffusers 17.

The functioning of the emergency aid model 1 is depicted as follows based on the initial emergency treatment of asystole referring to the FIGS. 2a)-d). In the FIGS. 2a)-d), it is shown partially the cross section of a part of the body 22 or a part of the left breastbone of a patient lying in supine with the parts of skin 23, the ribs 24 and a part of the heart 25 including a chamber 26. In the case, there are ribs 24, for more exact the third and the fourth rib, and the heart chamber for the right heart chamber of ventricle.

The first aider will localize the emergency aid model 1 in the range of the left breastbone in the about nearness of the heart 25 of the body 22, and switch on through the activation button 6. FIG. 2a) shows that the emergency aid model 22 is put on the body 22 with its base area of the housing 2 on the body. Through the sensor array 5 of the position determination apparatus 13 or through the infrared sensor, the position of the opening 4 in the base area of the housing 2 after switching on and in some embodiment forms of the emergency aid model 1 the position of the electrode 16, is determined in relation to the right heart chamber. Furthermore, the existence of a rib 24 between the opening 4 or the electrode 16 and the heart chamber 26 will be detected by the sensor array 5. The collected data by the sensor array 5 will be forwarded to the control unit 12 of the position determination apparatus 13, and there checked whether the acquired current position of the emergency aid model 1 on the body 22 or the position of the opening 4 or the electrode 16 in relation to the heart chamber 26 is suitable for the puncturing apparatus 9 getting into the final position where the electrode 16 contacts the heart 25. At the position of the emergency aid model 1 shown in the FIG. 2a), though the opening is located above the heart 25 as well as above the heart chamber 26, the penetration way of the puncturing apparatus 9 is prevented by a rib 24. This will be recognized by the control unit 12, and consequently the red light-emitting diode will light up, so that the first aider will be warned that getting the puncturing apparatus 9 into the final position is impossible at this position of the emergency aid model 1 or at the present position of the opening 4 or the electrode 16 in relation to the heart 25 or the heart chamber 26. Thus, the first aider knows that he has to move the emergency aid model 1 to the other site, upon which as described above the position of the opening 4 or the electrode 16 in relation to the heart 25 or the heart chamber 26 is determined once again by the sensor array 5 and checked by the position determination apparatus 13 if the puncturing apparatus 9 can be got to the final position or not. If it is not the case, the red light-emitting diode lights up once again in case it is extinguished before, and this process will be repeated so long until a suitable site for the emergency aid model 1 or a suitable position of the opening 4 or the electrode 16 in relation to the heart 25 or the heart chamber 26 is found.

FIG. 2b) shows that after the displacement from the unsuitable site shown in FIG. 2a), the emergency aid model 1 now is at a suitable position to get the puncturing apparatus 9 to the final position. Although the opening 4 is still located directly over the heart 25 and the heart chamber 26, the penetration way of the puncturing apparatus 9 into the heart 25 is no longer prevented by the rib 24. Based on the movable installation elements 3 that displace itself due to a suitable suspension in either one embodiment form or is correspondingly moved by the control unit 12 in the other embodiment form, the base area of the housing 2 adapts to the specific form of the body 22 in this position, whereby the emergency aid model 1 gets ensured laying on the body 22, whereby the emergency aid model 1 is additionally prevented from eventually shifting through the designed bonding material between the installation elements 3 and the skin 23 of the body 22. According to the data delivered by the sensor array 5, the control unit 12 realizes the suitability of the current position of the emergency aid model 1 or the position of the opening 4 or the electrode 16 in relation to the heart 25 or the heart chamber 26 for the puncturing apparatus 9 to get the final position, and thus switches on the green light-emitting diode 8 instead of lighting up the red light-emitting diode 7. This signal is for the first aider to initialize the penetration of the puncturing apparatus 9 into the body 22 or to the final position.

For this he presses the activation button 6. Now the control unit 12 steers the mechanics 15 that then pushes the puncturing apparatus 9 with its front-end part 14 firstly through the opening 4, and into the body 22 through the skin 23 and between the ribs 24. Since the front-end part 14 is a spiky end that easily splits the skin 23 as well as the below laying muscle and cartilage during the penetration of the front-end part 14 into the body 22, which makes it easier for the puncturing apparatus 9 to get into the body 22. Because the fluid pipe 20 is flexible and has enough length and furthermore because the electrical cable 19 is wound sectionwise and gives enough length through unwinding during the moving-out of the puncturing apparatus 9, the moving-out of the puncturing apparatus 9 from the housing 2 will not be prevented by neither the fluid pipe 20 nor the electrical cable. Hereby the process of the penetration of the front-end part 14 into the body 22 will be monitored by the position determination apparatus 13, whereby the position determination apparatus 13 upon demand intervenes the penetration process through the control unit 12 that steers correspondingly the mechanics 15. During the whole penetration process, the depth up to which the front-end part 14 is pushed into the body 22 will be determined through the ultrasonic sensor of the sensor array 5.

In the FIG. 2b), the puncturing apparatus 9 has already got so far into the body 22 that a front end of the puncturing apparatus 9 contacts or just touches the chamber wall of the heart 25. Principally, it is now already possible to stimulate the heart 25 with electrical impulses provided that the electrode 16 covers the spiky end of the puncturing apparatus 9. For some embodiment forms or operation modes of the emergency aid model 1, the present position of the puncturing apparatus 9 shown in FIG. 2b) is already the final position. In this case that the emergency aid model 1 is used to handle asystole, the outside touch of the electrode 16 to the heart 25 is not sufficient for the intended treatment because possibly after the first cardiac contraction trigged by the electrode 16 with electrical impulses, the position or the extension of the heart 25 changes, so that the mechanics 15 controlled by the control unit 12 pushes the puncturing apparatus 9 further forwards with the electrode 16 getting into the heart 25 or penetrating into the chamber wall as shown in FIG. 2c). Also the position of the puncturing apparatus 9 shown in FIG. 2c) can be considered as the final position for several embodiment forms or operation modes of the emergency aid model 1 since it is possible for the electrode 16 to give the impulses directly to the heart chamber wall.

In the case of the illustrated emergency aid model 1, the puncturing apparatus 9 will be further pushed until the front-end part 14 with the electrode 16 partly protrudes into the heart chamber 26 and partly remains within the chamber wall, as shown in FIG. 2d). For this present embodiment form of the emergency aid model 1, this corresponds to the final position of the puncturing apparatus 9. As soon as the puncturing apparatus 9 attains the final position depicted in the FIG. 2d), the control unit 12 ensures that the electrical impulses from the battery 10 via the electrical cable 19 and the electrode 16 will be applied to the heart 25. Furthermore, the control unit 12 steers the pump 21 in order to pump the drug from the drug reservoir 11 to the fluid pipe 20 and further to the gap between the front-end part 14 of the puncturing apparatus 9 where the drug will be sprayed through the diffusers 17 into the hear chamber 26. After successful treatment of the asystole, the puncturing apparatus 9 will be decoupled from the other parts of the emergency aid model 1 and fully separated from the housing 2. Now the patient can be transported to the hospital where the puncturing apparatus 9 will be removed from the body 22.

When the treatment of the asystole remains unexpectedly unsuccessful, thanks to its flat cover area the emergency aid model 1 the device can be used for application of chest compression. Hereby the puncturing apparatus 9 can supply electrical power to the heart as accompanying measures during the chest compression.

In the FIG. 3 is shown a simplified cross section of an alternative puncturing apparatus 27 to the puncturing apparatus 9. The puncturing apparatus 27 contains an outer tube element 28 and an inner electrode element 29, whereby the electrode 29 is put in the tube element 28 and telescopically movable out of the element 28. While the construction of the electrode element 29 mainly corresponds to that of the puncturing apparatus 9 of the emergency aid model 1 and thus not explained in details here, the tube element 28 is principally a stable tube equipped with a tapered front portion that protects the electrode element 29 during the penetration of the puncturing apparatus 27 into the body 22. Only after the greatest resistances for the penetration of the puncturing apparatus 27 into the body 22 are overcome, the electrode element 29 gets out of the tube element 28 and into the heart 25 without the tube element 28 getting into the heart 25.

The FIGS. 4a)-c) show in more details this via the emergency aid model 30 that differs from the emergency aid model 1 mainly by that it has the puncturing apparatus 27 shown in FIG. 3 instead of the puncturing apparatus 9.

By operating the emergency aid model 30 in order to get the puncturing apparatus 27 into the body 22 and get the final position, the stable tube element 28 will penetrate at first the outer layers of the body 22 like the skin 23, muscles and cartilage, which are difficult to be penetrated. During the penetration into the body 22, the electrode element 29, especially the not visible metal mesh at the front end serving as the electrode 31 remains protected by the tube element 28. FIG. 4a) shows this situation in which the tube element 28 gets so far into the body 22 that its front-end touches the chamber wall of the heart 25. Only when the resistant outer body layers are penetrated by the puncturing apparatus 27 or the tube element 28, the electrode element 29 gets out of the tube element 28 and the electrode element with its front-end portion and its arranged electrode 31 can now get into the heart 25 relatively without hindrance, which leaves the electrode element 29 comparably lower resistance. In the FIG. 4b), it is shown that the electrode element 29 has already penetrated into the heart 25 so far that the electrode 31 is within the heart chamber wall. For the first operation mode of the emergency aid model 30, this situation of the puncturing apparatus 27 already corresponds to the final position because the electrode 31 contacts the heart 25 so that it is possible to stimulate the heart 25 with electrical impulses. For the second operation mode of the emergency aid model 30, the electrode element 29 will be pushed further into the heart 25 until the front-end portion of the electrode element 29 with the electrode 31 partly protrudes in the heart chamber 26 with simultaneously partly localized within the chamber wall. In the second operation mode of the emergency aid model 30, this situation shown in the FIG. 4c) corresponds to the final position of the puncturing apparatus 27. Through the inserting part of the electrode 31 in the chamber wall, the heart 25 can be now stimulated, and simultaneously, the pharmaceutical treatment can be performed on the heart 25 by spraying the drug into the heart chamber 26 through the protruded part of the electrode element 29 in the heart chamber 26.

In the FIG. 5a)-c) is shown a further operation mode of the emergency aid model 32 with another puncturing apparatus 33. The puncturing apparatus 33 has a tube element 34 similar to that of the puncturing apparatus 27 shown in FIG. 3, and an electrode element 35 that is telescopically imbedded in the tube element 34 and can protrude out of the tube element; this electrode element is covered at the front-end with a metal mesh serving as the electrode 36. The electrode element 35 contains in addition to the electrode 36 a foldable electrode 37 that is kept in the inside of the electrode element 35 in a folded status and is shown in FIGS. 5b) and 5c), and can get the folded-out situation after getting out of the inside of the electrode element 35 and unfolding in umbrella-shaped. The foldable electrode 37 is essentially a metal mesh with a mechanism similar to that of an umbrella, whereby in the folded out status of the electrode 37, the metal mesh is stretched between single struts of the folding mechanism.

As in the emergency aid model 30 in the FIG. 4a)-c), for introducing the puncturing apparatus 33 into the body 22 in the emergency aid model 32 the tube element 34 will also be pushed at first into the body 22 till the chamber wall of the heart 25, whereby the electrode element 35 is hosted in the tube element 34 for its protection during the penetration of the tube element 34 into the body 22. Subsequently, the electrode element 35 gets out of the tube element 34. The electrode element 35 penetrates the chamber wall of the heart 25 until one part of it protruding in the heart chamber 26 as shown in FIG. 5a). Thereafter, the foldable electrode 37 gets out of the inside of the electrode element 35 and attains the folded-out situation where the foldable electrode shows a comparably big area as shown in FIG. 5b). In the FIGS. 5a) and 5b), the electrode 36 is fully in the inside of the heart chamber 26 due to a better clarity describing the operation mode of the emergency aid model 32; but in most cases, this electrode is partly inserted in the chamber wall in this mentioned situation. After folding-out of the foldable electrode 37, the electrode element 35 will be drawn back a bit again into the tube element 34 until the folded-out electrode 37 is attached to the inner wall of the heart chamber 26. At this present the electrode 36 as shown in FIG. 5c) is visible in the inside of the chamber wall while the foldable electrode 37 contacts the heart chamber 26 of the heart 25 through the inner wall. As shown in FIG. 5c), the situation of the puncturing apparatus 33 or the tube element 34 and the electrode element 35 inclusive of the electrode 37 indicates the final position of the puncturing apparatus 33 of the emergency aid model 32. Hereby the folded-out electrode 37 has the advantage like a barb that prevents shifting or pulling-out of the electrode element 35 from the heart 25 during the heart action triggered by the electrical impulses. It is possible at this final position of the puncturing apparatus 33 to stimulate the heart 25 with electrical impulses through the electrode 36 to the chamber wall or through the foldable electrode 37 to the inner wall of the heart chamber 26. For this, the heart 25 can be supplied with different or the same electrical impulses by the electrode 36 and the foldable electrode 37 simultaneously or alternatingly, or by just one of the both electrode 36 or 37, depending upon situations. At the same time, drugs can be delivered into the heart chamber 26 via the front-end of the puncturing apparatus 33.

A further embodiment form of the puncturing apparatus is that the foldable electrode 37 is replaced with a kind of expandable or inflatable electrode. In the FIGS. 6a) and 6b) it is shown that the operation mode of the emergency aid model 38 with such kind of the puncturing apparatus 39. The puncturing apparatus 39 also contains a tube element 40 and an electrode element 41 telescopically retracted and included in the tube element 40; and the front-end portion of the electrode element 41 is extensively covered with a metal mesh as electrode 42. Furthermore, the electrode element 41 has a balloon 43 included in the inside of the electrode element 41 with a folded status, and the outer surface of the balloon as well as the front-end portion of the electrode element 41 is covered by a metal mesh. The balloon 43 can be filled with water and thus inflated by a water reservoir that is not visible in FIGS. 6a) and 6b) and equipped in the emergency aid model 38, whereby the balloon extends at the front-end of the electrode element 41 out of its inside.

Similar to the puncturing apparatus 33 of the emergency aid model 32, in the emergency aid model 38 the puncturing apparatus 39 also penetrates at first with its tube element 40 into the chamber wall of the heart 25 of the body 22; subsequently, the electrode element 41 gets out of the tube element 40 and penetrates the chamber wall until its front portion protruding in the heart chamber 26 as for the tube element 34 of the puncturing apparatus 33 shown in FIG. 5a). Thereafter, the water will be pumped from the water reservoir into the folded balloon within the electrode element 41, then the balloon expands so that it gets out of the inside of the electrode element 41. FIG. 6a) shows the situation immediately after the filling of the balloon 43 that is fully kept in the inside of the heart chamber 26. The electrode element 41 will then draw back a bit into the tube element 40 until the puncturing apparatus 39 takes its final position as shown in FIG. 6b), where the expanded balloon 43 attaches the inner wall of the heart chamber 26, which functions as a mechanic assurance against shifting of the electrode element 41 or an unintended pulling-out of the electrode element 41 out of the heart 25. Now as in the case of the emergency aid model 32, an electrical contact between the electrode 42 inserted within the chamber wall and the chamber wall on one side and between the balloon 43 and the inner wall of the heart chamber 26 on the other side. Therefore, as described in FIG. 5c), the same or different electrical impulses can be applied to the heart 25, alternatingly, simultaneously or in single through the electrode 42 and the balloon 43.

An amplified cross section of the balloon 43 is shown in FIG. 7. It is shown that the balloon 43 principally contains an expansion element 44 that is wrapped with a membrane 45, and on the outer surface of the membrane is a metal mesh taking the function of an electrode. The membrane 45 made of silicon or polyethylene is given a number of even distributed openings 46 over the surface. To fill the balloon 43, the water will be introduced into the inside of the expansion element 44; subsequently, this element swells up and its volume enlarges. After filling the balloon 43 or the expanding of the expansion element 44, a drug will be inlet between the expansion element 44 and the membrane 45, which diffuses over the expansion element 44 and gets out of the opening 46 so that the drug will be sprayed evenly into the inside of the heart chamber 26.

Finally, in the FIGS. 8a) and 8b) is shown the operation mode of a further emergency aid model 47. The emergency aid model 47 has a puncturing apparatus 48 that contains an oblong tube element 49 similar to those described above and a balloon 50 similar to the above-mentioned expansion element 44; however, the puncturing apparatus 48 lacks an electrode element as that in the above-described examples. The balloon 50 is included in the tube element 49 in a folded, not expanded status. It is inflatable with air, which is provided by a container in the housing of the emergency aid model 47. Differently from the above-described embodiment examples, the tube element 40 does not completely penetrate to the wall of the heart 25 but keeps a distance to this wall when the puncturing apparatus 48 penetrates into the body 22. This is shown in FIG. 8a). Once the balloon 50 is inflated, it gets out of the tube element 49 and molds itself to the outer surface of the chamber wall of the heart 25. Instead of attaching the inner wall of the heart chamber 26 as in the previous embodiment example, the balloon 50 for the present example attaches to the outside of the heart 25 and gets contact to the heart 25, as shown in FIG. 8b. The electrical impulses can be given to the heart through the balloon 50 from the outer chamber wall.

The above-mentioned emergency aid models are principally intended for automated application. One or more such emergency aid models can also be used as a part of a complex emergency aid equipment that provides more functions.

Such a multifunctional apparatus 51 is shown in FIG. 9. The multifunctional apparatus has a housing 52 with a display 53, two defibrillator electrodes 54 attached to but separable from the housing 52 as well as an emergency aid model 55 from the present invention, which is also attached to and separable from the emergency aid equipment 51. Through the display 53, the multifunctional emergency aid equipment can be operated, which can be used selectively as defibrillator through the defibrillator electrodes 54 or as emergency treatment of asystole through the emergency aid model 55. In this case, the control of the emergency aid model 55 is taken over by the control unit that is included in the housing 52 and is not visible in FIG. 9. In order to enable the communication between the control unit and the emergency aid model 55 after detached from the housing 52, there will be equivalently a cable connection or a wireless connection, e.g. Bluetooth or a comparable connection between the emergency aid model 55 and the control unit.

All the above-described emergency aid models 1, 30, 32, 38, 47, 55 can be modified, so that instead of the internal drug reservoir 2 included in housing 2 and case to case also instead of the pump 21 or in addition to the drug reservoir 11 and the pump 21, they can have a connection through which they can be connected to an external drug reservoir or any external drug feed, e.g. one or more hoses. Through these connections, the emergency aid model 1, 30, 32, 38, 47, 55 can be supplied externally with drugs, which can get into the heart 25 via the already described functional modes of each puncturing apparatus 9, 27, 33, 39, 48. For this application, either an external control unit will be taken or the control unit of each emergency aid model 1, 30, 32, 38, 47, 55 is equipped for controlling the drug supply. Furthermore, besides the mentioned drug reservoir 11 any further drug reservoirs can be included, which are filled with a single drug each. The drugs of these drug reservoirs can be transported separately from each pump through the fluid pipe 20 or the control unit 12 gives the instruction making a drug mixture in relation to their respective amounts and delivering it to the fluid pipe 20, which can be done, for instance, by controlling each corresponding pump, for instance; hence, the heart 25 can be treated either with each single drugs or with a drug mixture with the respective drugs upon the demand. Furthermore, instead of the pump 21 a piston with pneumatically driving can be preferred for the drug reservoir 11, which pushes the drug out of the drug reservoir 11 into the fluid pipe 20. Preferably, the hub of the piston is controlled by the control unit 12.

When the stimulation of the heart 25 with electrical impulses is successful, the heart begins to move with contractions and expansions again. Hereby there exists a difference in distance up to 2 cm to the heart chamber wall. With these movements there is a possibility that the puncturing apparatus dissociates from the heart and the contact between the electrode and the heart will be broken. This can be avoided by using a suitable tracking system, which ensures that the puncturing apparatus tracts the movements of the heart so that the contact between the electrode and the heart can be maintained even after the initialization of the heart movements.

In the FIGS. 10a) and 10b) there are examples for this kind of tracking system, particularly for such embodiment forms of the emergency aid models where an internal fixing of the puncturing apparatus in the heart 25 is intended. This is the case, for instance for the emergency aid model 32 and 38 shown in the FIGS. 5c) and 6b). Without limitation of the generality, the tracking system and its function are described as follows based on the electrode element 41 of the puncturing apparatus 39 of the emergency aid model 38.

It is shown in FIGS. 10a) and 10b) that the electrode element 41 penetrates the breastbone 56 and the muscle 57 of the patient, whereby the electrode 42 is already within the heart wall and the balloon 43 inflated as already described in FIG. 6b). At the end of the electrode element remote to the heart 25, there is a circular plate element 58 attached and fixed to the electrode element 41. Between the plate element 58 and the corresponding inner wall of the housing 2 of the emergency aid model 38 with the opening 4, there are coil springs 59 intended, which are strained by the plate element 58 when the electrode element 41 gets out of the opening 4. Briefly, there are four such coil springs 59 attached to the electrode element 41, certainly only two visible in the FIG. 10a). Upon the strain of the coil springs 59, a restoring force will be applied to the extended electrode element 41, which retracts the electrode element 41.

Once the heart 25 beats again after the electrical stimulation and causes contraction movements, the electrode element 41 will be pulled out from the housing 2 of the emergency aid model 38 upon the balloon 43 attached to the inner chamber wall. Hereby the coil springs will be more strined and the restoring force increases. During the subsequent relaxation of the heart 25, this restoring force ensures that the electrode element 41 retracts back to the housing 2 again. Thus, the coil springs 59 and the balloon 43 attached to the inner wall of the heart chamber ensure that the electrode 42 does not loss the contact to the heart 25 during the movements of the heart 25 with contraction and expansion.

In the embodiment example shown in the FIG. 10b) is only one coil spring intended, which is penetrated through by the electrode element 41 instead of surrounding it. The operation mode shown in the FIG. 10b) is so far analogue to that of the embodiment example shown in FIG. 10a).

Instead of the coil springs shown in FIGS. 10a) and 10b), the restoring force resulted from straining can also be attained from the tracking system through springs, which can be stretched during the getting out of the puncturing apparatus, and the restoring force can be applied to the puncturing apparatus upon its stretching. For example, the puncturing apparatus can be equipped with one or more spiral springs or with one or more tension springs or both of them.

An emergency aid model with tracking system that has a spiral spring 61 is shown in FIG. 11a) with the bird view. The spiral spring 61 is fixed within the housing 2 and connected to the puncturing apparatus 41 with its end. When the puncturing apparatus 41 gets out, the spiral spring 61 fixed to the housing 2 and connected with the puncturing apparatus 41 will be stretched, whereby the restoring force is applied to the pulled out puncturing apparatus 41; the farther the puncturing apparatus gets out, the greater the restoring force. In contrast, in the emergency aid model shown in FIG. 11b), the spiral spring 61 is replaced by a tension spring, for instance, a coil spring. Otherwise, the operation mode of the embodiment example shown in FIG. 10b) is analogue to that of the embodiment example shown in FIG. 11a).

### LIST OF REFERENCE SIGNS

1. Emergency aid model
2. Housing
3. Installation element
4. Opening
5. Sensor array
6. Activation button
7. Red light-emitting diode
8. Green light-emitting diode
9. Puncturing apparatus
10. Battery
11. Drug reservoir
12. Control unit
13. Position determination apparatus
14. Front-end portion
15. Mechanics
16. Electrode
17. Diffuser
18. Rear end
19. Electrical cable
20. Fluid pipe
21. Pump
22. Body
23. Skin
24. Ribs
25. Heart
26. Heart chamber
27. Puncturing apparatus
28. Tube element
29. Electrode element
30. Emergency aid model
31. Electrode
32. Emergency aid model
33. Puncturing apparatus
34. Tube element
35. Electrode element
36. Electrode
37. Foldable electrode
38. Emergency aid model
39. Puncturing apparatus
40. Tube element
41. Electrode element
42. Electrode
43. Balloon
44. Expansion element
45. Membrane
46. Opening
47. Emergency aid model
48. Puncturing apparatus
49. Tube element
50. Balloon
51. Multifunctional apparatus
52. Housing
53. Display
54. Defibrillator electrode
55. Emergency aid model
56. Breastbone
57. Muscle
58. Plate element
59. Coil spring
60. Coil spring
61. Spiral spring
62. Tension spring

1. A device for stimulating a given tissue with electrical impulses that is abuttable against body surfaces of organisms, comprising:
at least one puncturing apparatus, which has at least one electrode, the puncturing apparatus provided to penetrate into an organism after abutting the device against the body surface of the organism, and to take a final position where the electrode contacts the tissue of the organism;
at least one position determination apparatus provided to determine a position of at least one part of the device abutting against the body surface in relation to at least one part of the tissue, and subsequently to check the determined position whether it is suitable for transferring the puncturing apparatus into the final position; and
at least one signal output element for outputting signals, wherein the device is configured to output at least one signal dependent on the result of the check of the determined position.

2. The device according to claim 1, wherein the position determination apparatus recognizes the determined position as suitable if the determined position is within at least one pre-set position range or if a route from an intended injection site for the puncturing apparatus to the tissue is free of obstacles.

3. The device according to claim 1, further comprising at least one sensor or at least one sensor array (5) or at least one ultrasonic sensor or at least one infrared sensor or at least one force sensor or at least one depth sensor or at least one magnetic position sensor or at least one laser sensor or at least one laser-emitting diode or at least one gas sensor or at least one skin resistance sensor or at least one sensor measuring impedance.

4. The device according to claim 1, wherein the puncturing apparatus comprises at least one cutting element for cutting the organism in order to make it easier for the puncturing apparatus to get into the organism.

5. The device according to claim 1, wherein the puncturing apparatus has at least two elements that are slidable in a telescope-like way into one another and apart.

6. The device according to claim 1, wherein the electrode covers or forms at least a part of an outer surface of the puncturing apparatus;
optionally, wherein the puncturing apparatus comprises at least one foldable element whereby the electrode covers or forms at least a part of the outer surface of the foldable element;
optionally, wherein the puncturing apparatus comprises at least one expandable element whereby the electrode covers or forms at least a part of the outer surface of the expandable element;
optionally, wherein the puncturing apparatus comprises at least one extendable element whereby the electrode covers or forms at least a part of the outer surface of the extendable element.

7. The device according to claim 1, further comprising at least one drug feed for supplying the tissue with at least one drug, and at least one drug reservoir coupled to the drug feed for receiving at least one drug; and
optionally, at least one drug inlet coupled with the drug feed for getting at least one drug from outside the device.

8. The device according to claim 1, further comprising at least one movable abutting element for abutting the device against body surfaces of organisms.

9. The device according to claim 1, wherein a depth for penetration of the puncturing apparatus into the organism is adjustable.

10. The device according to claim 1, further comprising at least one control unit configured for controlling the delivery of electrical impulses to the tissue or checking the determined position or the output of signals or the penetration of the puncturing apparatus into the organism or the transfer of the puncturing apparatus to the final position and/or the contact of the electrode to the tissue or the folding-out of the foldable element or the expansion of the expandable element or the supply of drugs to the tissue or the positioning adjustment of the abutting element against an outer form of the body surface.

11. The device according to claim 1, further comprising a housing, wherein the puncturing apparatus can be decoupled from the housing.

12. The device according to claim 1, further comprising a tracking system for tracking the final position of the puncturing apparatus when the tissue performs movements.

13. A multifunctional apparatus having a device according to claim 1, further comprising at least one defibrillator or at least one defibrillator electrode or at least one cardiac pacemaker.
